(19) United States
(12) Patent Application Publication
(10) Pub. No.: US 2005/0113692 A1
(43) Pub. Date: May 26, 2005

(54) Structure containing the conductive medium for the source of the external high-power focusing ultrasonic treatment

(76) Inventors: Shenxu He, Beijing (CN); Jinsheng Yu, Beijing (CN); Jiang Lan, Beijing (CN); Xiaodong Wu, Beijing (CN)

Correspondence Address: BURNS DOANE SWECKER & MATHIS L L P, POST OFFICE BOX 1404, ALEXANDRIA, VA, 22313-1404, US

(21) Appl. No.: 10/494,269

(22) PCT Filed: Mar. 15, 2002
(86) PCT No.: PCT/CN02/00167

(30) Foreign Application Priority Data
Nov. 5, 2001 (CN) 01134483.0

(51) Int. Cl. A61N 7/00

(52) U.S. Cl. 600/439; 601/2; 600/459

(57) ABSTRACT

The present invention provides a containing means for receiving the transmission medium for use With an ultrasonic wave source of an extracorporeal HIFU therapeutic apparatus, wherein the containing means for transmission medium is a semispheric membrane with a seating arragnment on an upper open end thereof for sealing connecting the containing means to the ultrasonic wave source, so that the membrane closes the ultrasonic wave emitting surface. The membrane is made from a material with an acoustic impedance close to that of the water, lower ultrasound transmission loss, perfect heat conductivity and good elasticity, such as latex, natural rubber, silicon rubber etc. The containing means can be used in the case that the ultrasonic wave source is mounted above the bed surface. It is easy to make the ultrasonic wave source rotated and tilted so that the inclining angle of the ultrasonic wave source may be adjusted as required so as to adapt to the variety of treatment requirements.

### FIELD OF THE INVENTION

The present invention relatcs to a containing means for use in a medical apparatus, especially to a containing means for receiving the transmission medium for use in a wave source of an extracorporeal high intensity focus ultrasonic wave (HIFU) therapeutic apparatus, i.e. a container for use in a wave source of an extracorporeal HIFU therapeutic apparatus.

### BACKGROUND OF THE INVENTION

At present, the existing HIFU therapeutic apparatus generally includes the following means: a HIFU source and a driving circuit thereof for providing the HIFU; a locating system for searching the object to be treated and moving it to the focal point of the ultrasonic wave transducer; a high intensity ultrasonic wave (HIU) transmitter and a processing system for the transmission medium. Since the ultrasonic wave adapted to the HIFU must be transmitted into the patient body through a special transmission medium (such as deaerated water) , a container for receiving the transmission medium in front of the emitting surface of the HIFU source and means for filling, discharging and processing the transmission medium are necessary.

All the exiting extracorporeal HIFU therapeutic apparatus for clinical applications use the arrangement of a “lower-mounted ultrasonic wave source”, that is, the HIFU source is positioned below the bed surface of the therapeutic apparatus and the ultrasonic wave is transmitted into the patient body through the deaerated water contained in a container for receiving the transmission medium such as a water tank opening into the bed surface. For most of the patients, when treating the tumors in the abdominal cavity and pelvic cavity by HIFU, only from the front can the ultrasonic wave be transmitted into the human body because the ultrasonic wave may be blocked by the spine and the pelvis when transmitted from the back of the patient. Therefore, when being treated by the lower-mounted ultrasonic wave source, most of patients have to be in a pronation pose. The disadvantages of the pronation pose are the following: the physical strength dissipation of the patient will be much more and thus the patient can not bear a long time treatment; when in the pronation pose of the patient, the abdominal wall and the intestinal canals will sag under the gravity action while the positions of the tumors are relatively fixed, which will cause the distance from the skin to the tumors to substantially increase so as to cause the increase of the ultrasound attenuation along the transmission path of the HIFU, and the ultrasound attenuation will reduce the sound intensity at the focal point and affect the therapy effect; the gas in the stomach and intestine will disturb the locating effected by the B-Mode Ultrasonoscope.

If the extracorporeal HIFU therapeutic apparatus is provided with an upper-mounted ultrasonic wave source (the ultrasonic wave source is mounted above the bed surface of the therapeutic apparatus), the above-mentioned disadvantages of the lower-mounted wave source will be overcome so that the tired degree of the patient and the difficulty of locating will be substantially reduced, and the therapy effect will be improved. However, if the waste source is mounted above the bed surface, it is difficult to select the containing means for containing the transmission medium to efficiently transmit the high intensity ultrasonic wave into the patient body, which is the reason why the conventional HIFU therapeutic apparatus do not use an upper-mounted ultrasonic wave source. If the containing means is an open water tank, the water tank must be very big and thereby the amount of the deaerated water for transmitting the ultrasonic wave will correspondingly increase, so that the deaerated water will rapidly deteriorate due to the excessive contact area between the deaerated water and air. Therefore it is very difficult to technically use a containing means for transmission medium such as an open water tank for an upper-mounted ultrasonic wave source.

Further, in the case of a lower-mounted ultrasonic wave source, if the containing means for transmission medium is an open water tank and filled with deaerated water the position of the water tank will be greatly limited and shall be only in horizontal position. Otherwise, the medium in the containing means sill overflow from one side thereof. Therefore, the inclining angle of the ultrasonic wave source is very limited so that the tumors behind the costal margin, xiphisternum and pubis can not be treated to meet a variety of treatment requirements.

### SUMMARY OF THE INVENTION

The object of the present invention is to overcome the above-mentioned disadvantages by providing a containing means for receiving the transmission medium for use with a ultrasonic wave source of an extracorporeal HIFU therapeutic apparatus. Such containing means can be used for the ultrasonic wave source mounted above the bed surface, and the high intensity ultrasonic wave (HIU) can be efficiently transmitted into the patient body though the medium while the inclining angle of the ultrasonic wave source can be adjusted as required so as to meet the variety of treatment requirements.

In order to fulfill the above-mentioned object, the present invention provides a containing means for receiving the transmission medium for use with a ultrasonic wave source of an extracorporeal HIFU therapeutic apparatus, in which the ultrasonic wave source of the therapeutic apparatus has a ultrasound emitting surface, wherein the containing means for transmission medium is a semispheric membrane with a sealing arrangement on an upper open end thereof for sealingly connecting the containing means to the ultrasonic wave source, so that the membrane closes the ultrasound emitting surface of the wave source. The transmission medium is filled between the membrane and the emitting surface.

The means of the present invention can further include the following features:

The sealing arrangement may be an annular sealing flange which is pressed against an outer rim of the ultrasonic wave source through a pressing ring by thread fastening so as to close the ultrasonic wave emitting surface of the ultrasonic wave source.

The membrane may be made from a material with an acoustic impedance similar to that of the water, with lower ultrasound transmission loss, perfect heat conductivity and good elasticity, such as latex, natural rubber, silicon rubber etc.

The ultrasonic wave transmission medium may be deaerated water. Preferably, the outer side of the membrane is coated with an ultrasound couplant.

Since the containing means for transmission medium according to the present invention uses the membrane to close the emitting surface of the upper-mounted ultrasonic wave source, a “water bag” is formed by filling the transmission medium such as deaerated water between the membrane and the emitting surface. The membrane coated with the ultrasound couplant on the outer side thereof is placed on the skin of the patient and keep in rood contact with the patient skin, which will efficiently transmit the HIU into the patient body so as to achieve the HIFU therapy in the case that the ultrasonic wave source is mounted above the bed surface.

At the same time, since the upper-mounted ultrasonic wave source is closed and it is easy to be rotated and tilted, the inclining angle of the ultrasonic wave source may be adjusted as required so as to adapt to the variety of treatment requirements. The ultrasonic wave source can perform a translation movement with respect to the patient and can be adjusted to a ccitain therapy deep degree as required.

### BRIEF DESCRIPTION OF DRAWINGS

The present invention will be described in detail by the preferred embodiments with reference to the following drawings, wherein,

FIG. 1 is a structural schematic view of an embodiment according to the present invention;

FIG. 2 is a sectional view of the embodiment shown in the FIG. 1;

FIG. 3 is a structural schematic view of an ultrasonic wave source with a containing means for transmission medium shown in the FIG. 1 mounted therefor;

FIG. 4 is a structural schematic view showing that the containing means for transmission medium is connected to the ultrasonic wave source;

### THE BEST MODE OF THE EMBODIMENTS

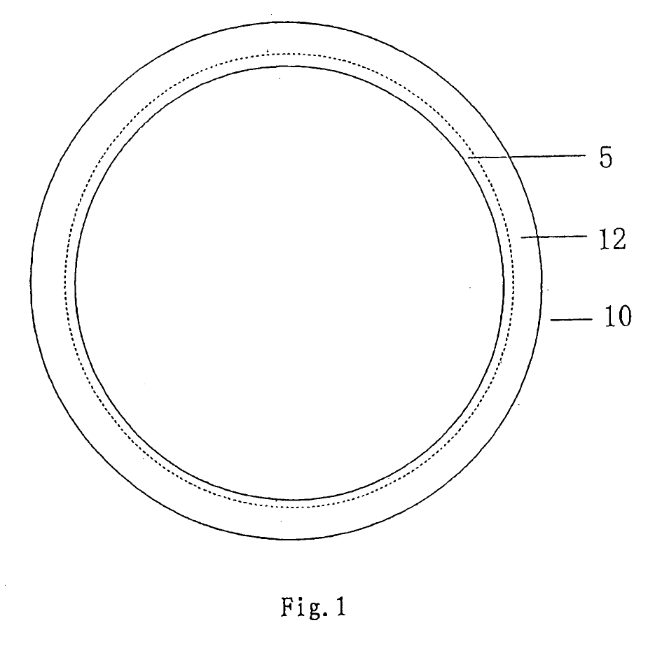

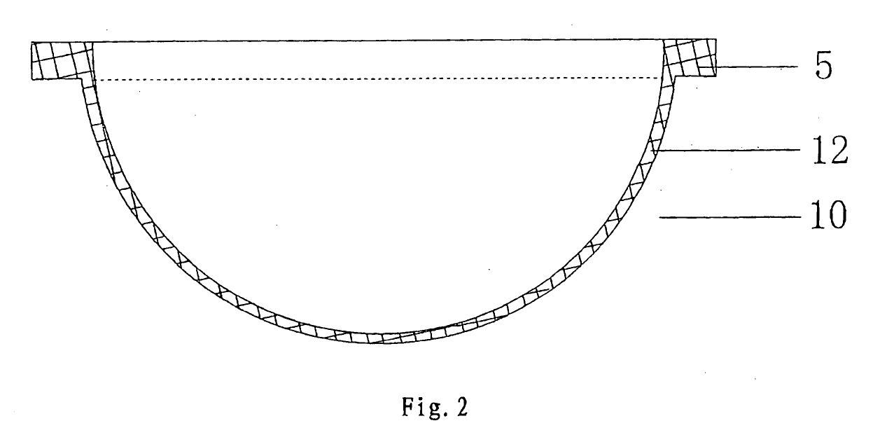

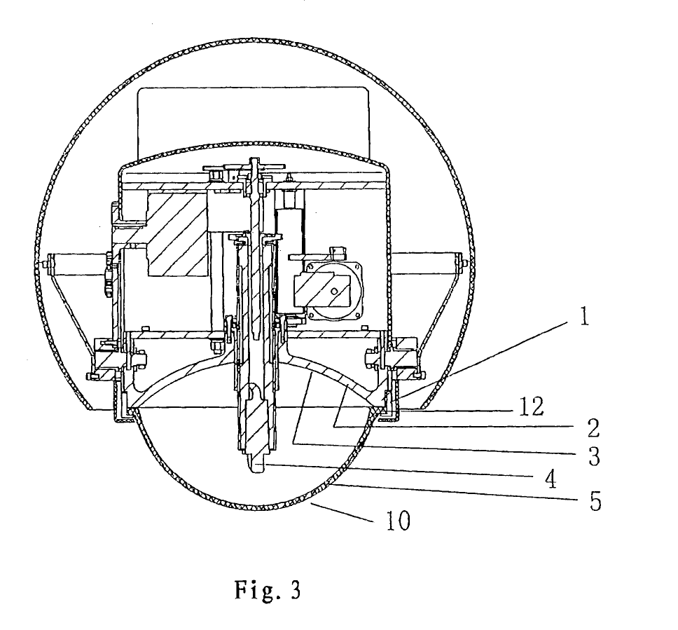

FIGS. 1 and 2 show one embodiment of a containing means 10 for receiving transmission medium according to the present invention. The ultrasonic wave source 2 of the therapeutic apparatus has an ultrasound emitting surface 3, wherein the containing means 10 for transmission medium is a semispheric membrane 5 with a sealing arragnment 12 on an upper open end thereof for sealingly connecting the containing means 10 to the ultrasonic wave source 2, so that membrane 5 closes the ultrasound emitting surface 3 of the source 2, as shown in FIG. 3. The transmission medium (not shown) is filled between the membranes and the emitting surface 3. The sealing arrangement 12 may be an annular sealing flange.

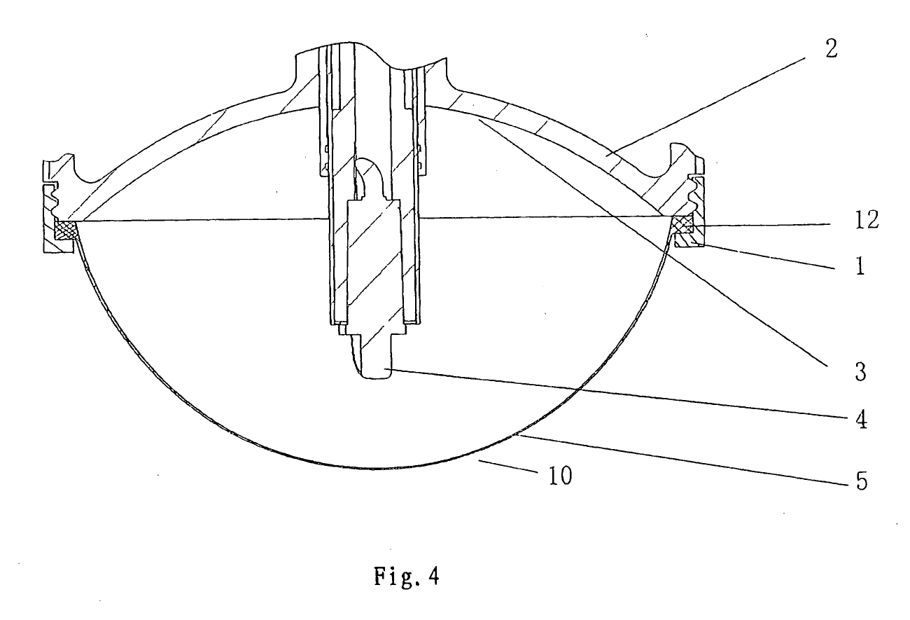

FIG. 3 shows an upper-mounted ultrasonic wave source 2 with the membrane-type containing means 10 for transmission medium mounted thereon. FIG. 4 shows the connection relationship between the membrane-type containing means 10 for transmission medium and the ultrasonic wave source 2. The reference number 1 designates the pressing ring for connecting the membrane 5 to the wave source 2, the reference number 2 designates the power ultrasonic wave source, the reference number 4 designates a probe head of B-Mode Ultrasonoscope which is mounted for the ultrasonic wave source 2. In the embodiment, the sealing arrangement 12 of the membrane 5 is pressed against an outer flat rim of the ultrasonic wave source 2 through a pressing ring 1 by thread fastening so as to close the ultrasonic wave emitting surface 3 of the ultrasonic wave source 2.

The ultrasound transmission medium may be deacrated water. Preferably, the outer side of the membrane 5 is coated with an ultrasound couplant.

The membrane 5 may be made from a material with an acoustic impedance close to that of the water, with lower ultrasound transmission loss, perfect heat conductivity and good elasticity, such as latex, relatively soft natural rubber, silicon rubber etc.

The containing means 10 for transmission medium according to the present invention uses the membrane 5 made in a special form and from a special material to close the emiting surface 3 of the upper-mounted ultrasonic wave source 2, and a “water bag” is formed by filling the ultrasonic transmission medium such as deacrated water between the membrane 5 and the emitting surface 3. The membrane 5 coated with the ultrasound couplant on the otter side thereof keeps in good contact with the patient skin, which will efficiently transmit the HIU into the patient body so as to achieve the HIFU therapy in the case that the ultrasonic wave source is mounted above the bed surface.

Since the membrane is in a desired shape and made of a suitable material, the membrane 5 can be adapted to the variety of treatment requirements of HIFU therapy for clinical applications. The ultrasonic wave source 2 can perform a translation movement with respect to the patient and can be adjusted to certain therapy deep degree as required. The ultrasound coupling between the membrane 5 and skin is perfect, the ultrasound loss and the heat conductivity of the membrane 5 can meet the requirements of the HIFU therapy and can efficiently transmit the HIU into the patient body. The deaerated water received in the containing means will not deteriorate because the deaerated water hardly contacts the air, and its size can be reduced greatly.

With the upper-mounted ultrasonic wave source 2 with the above-mentioned containing means 10 for transmission medium, the patient can be treated in a supination pose. In this case, the distance from the skin to the tumors may be substantially reduced under the gravity action and the compression force of the water bag awhile the gas in the intestine will be extruded as more as possible. Therefore the therapy effect on the tumors in abdomen, especially on the adenocarcinoma of pancreas and the lymphnode metastatic carcinoma in abdomen will be increased greatly.

Since the upper-mounted source is closed, the upper-mounted ultrasonic wave source can be freely rotated and inclined so that the tumors behind the costal margin, xiphistemum and pubis can be treated, which extend the treatment range of the HIFU therapeutic apparatus.

Reduction of the distance from the skin to the tumors due to the compression force of the water bag may reduce the disturbing of the gas so that realtime monitoring of the B-Mode Ultrasonoscope will be clearer during the treatment.

1. A containing means for receiving the transmission medium for use with an ultrasonic wave source (2) of an extracorporeal HIFU therapeutic apparatus, in which the ultrasonic wave source (2) of the therapeutic apparatus has a ultrasonic wave emitting surface (3), wherein the containing means for transmission medium is a semispheric membrane (5) with a sealing arragnment (12) on an upper open end thereof for sealingly connecting the containing means (10) to the ultrasonic wave source (2), so that membrane (5) closes the ultrasonic wave emitting surface (3) of the wave source (2), the ultrasonic wave transmission medium is filled between the membrane (5) and the emitting surface (3).

2. A containing means for receiving the transmission medium for use with an ultrasonic wave source of an extracorporeal HIFU therapeutic apparatus according to claim 1, wherein the sealing arrangement (12) is an annular sealing flange.

3. A containing means for receiving the transmission medium for use with an ultrasonic wave source of an extracorporeal HIFU therapeutic apparatus according to claim 1, wherein the ultrasonic wave transmission medium is deaerated water.

4. A containing means for receiving the transmission medium for use with an ultrasonic wave source of an extracorporeal HIFU therapeutic apparatus according to claim 1, wherein the outer side of the membrane is coated with a ultrasound couplant.

5. A containing means for receiving the transmission medium for use with an ultrasonic wave of an extracorporeal HIFU therapeutic apparatus according to claim 1, wherein the sealing arrangement (12) of the membrane (5) is pressed against an outer rim surface of the ultrasonic wave source (2) through a pressing ring (1) by thread fastening so as to close the ultrasonic wave emitting surface (3) of the ultrasonic wave source (2).

6. A containing means for receiving the transmission medium for use with an ultrasonic wave source of an extracorporeal HIFU therapeutic apparatus according to claim 1, wherein the membrane (5) is made from a material with an acoustic impedance close to that of the water, with lower ultrasound transmission loss, perfect heat conductivity and good elasticity.

7. A containing means for receiving the transmission medium for use with an ultrasonic wave source of an extracorporeal HIFU therapeutic apparatus according to claim 6, wherein said material is latex.

8. A containing means for receiving the transmission medium for use with an ultrasonic wave source of an extracorporeal HIFU therapeutic apparatus according to claim 6, wherein said material is a softer natural rubber.

9. A containing means for receiving the transmission medium for use with an ultrasonic wave source of an extracorporeal HIFU therapeutic apparatus according to claim 6, wherein said material is a silicon rubber.
